(19) United States
(12) Patent Application Publication
(10) Pub. No.: US 2013/0331787 A1
(43) Pub. Date: Dec. 12, 2013

(54) BACTERIA-RETAINING MEDICAL VALVE

(71) Applicant: Menahem Kraus, Rehovot (IL)

(72) Inventor: Menahem Kraus, Rehovot (IL)

(21) Appl. No.: 13/983,095

(22) PCT Filed: Feb. 2, 2012
(86) PCT No.: PCT/IL12/00059

§ 371 Date: Aug. 1, 2013

Related U.S. Application Data
(60) Provisional application No. 61/462,407, filed on Feb. 3, 2011.

(51) Int. Cl. A61M 39/26

(52) U.S. Cl. CPC A61M 39/26
USPC 604/126

(57) ABSTRACT

A bacteria-retaining medical valve including a housing defining a liquid inlet and a liquid outlet, a valve located within the housing downstream of the liquid inlet and a bacterial filter assembly located within the housing downstream of the valve and upstream of the liquid outlet, the bacterial filter assembly including hydrophilic bacterial impermeable passageways which are configured to permit liquid passage therethrough and prevent bacterial passage therethrough and air passage therethrough and hydrophobic bacterial impermeable passageways which are configured to permit air passage therethrough and prevent bacterial passage therethrough and liquid passage therethrough.

### REFERENCE TO RELATED APPLICATIONS

Reference is made to U.S. Provisional Patent Application Ser. No. 61/462,407, filed Feb. 3, 2011 and entitled “FILTER CONTAINING NEEDLELESS VALVE”, the disclosure of which is hereby incorporated by reference and priority of which is hereby claimed pursuant to 37 CFR 1.78(a) (4) and (5)(i).

### FIELD OF THE INVENTION

The present invention relates to medical valves and more particularly to bacteria-retaining medical valves.

### BACKGROUND OF THE INVENTION

The following publications are believed to represent the current state of the art:

U.S. Pat. Nos. 4,479,874; 4,797,259; 5,139,668; 5,401,403; 5,569,209; 5,851,390; 5,961,326; 6,736,138; 6,743,214 and 7,179,378.

### SUMMARY OF THE INVENTION

The present invention seeks to provide a bacteria-retaining medical valve or an improved bacterial filter for medical use.

There is thus provided in accordance with a preferred embodiment of the present invention a bacteria-retaining medical valve including a housing defining a liquid inlet and a liquid outlet, a valve located within the housing downstream of the liquid inlet and a bacterial filter assembly located within the housing downstream of the valve and upstream of the liquid outlet. The bacterial filter assembly includes hydrophilic bacterial impermeable passageways which are configured to permit liquid passage therethrough and prevent bacterial passage therethrough and air passage therethrough and hydrophobic bacterial impermeable passageways which are configured to permit air passage therethrough and prevent bacterial passage therethrough and liquid passage therethrough.

Preferably, at least an upstream part of the bacterial filter assembly reduces the pressure of liquid reaching at least a downstream part of the bacterial filter assembly and at least some of the hydrophobic bacterial impermeable passageways are located so as to prevent air buildup within the bacterial filter assembly at at least some of the hydrophilic bacterial impermeable passageways at which the liquid pressure is substantially reduced, which would otherwise block liquid flow therethrough.

In accordance with a preferred embodiment of the present invention the hydrophobic bacterial impermeable passageways are formed in an air vent filter which is upstream of the hydrophilic bacterial impermeable passageways.

Preferably, the bacterial filter assembly includes a folded bacterial filter element which, when folded, has multiple, non co-planar liquid and air passage surfaces. Additionally or alternatively, the bacterial filter assembly includes a spiral wound filter element and spacer. Alternatively or additionally, the bacterial filter assembly includes an envelope formed of a filter substrate which encloses a spacer and is formed with a single aperture.

In accordance with a preferred embodiment of the present invention the bacterial filter assembly includes a bundle of mutually spaced hollow fibers.

There is also provided in accordance with another preferred embodiment of the present invention a bacteria-retaining medical filter including a housing defining a liquid inlet and a liquid outlet and a bacterial filter assembly located within the housing downstream of the liquid inlet and upstream of the liquid outlet, the bacterial filter assembly including hydrophilic bacterial impermeable passageways which are configured to permit liquid passage therethrough and prevent bacterial passage therethrough and air passage therethrough and hydrophobic bacterial impermeable passageways which are configured to permit air passage therethrough and prevent bacterial passage therethrough and liquid passage therethrough, and wherein at least an upstream part of the bacterial filter assembly reduces the pressure of liquid reaching at least a downstream part of the bacterial filter assembly and at least some of the hydrophobic bacterial impermeable passageways are located so as to prevent air buildup within the bacterial filter assembly at at least some of the hydrophilic bacterial impermeable passageways at which the liquid pressure is substantially reduced, which would otherwise block liquid flow therethrough.

Preferably, the bacterial filter assembly includes a folded bacterial filter element which, when folded, has multiple, non co-planar liquid and air passage surfaces. In accordance with a preferred embodiment of the present invention the bacterial filter assembly includes a spiral wound filter element and spacer. Additionally or alternatively, the bacterial filter assembly includes an envelope formed of a filter substrate which encloses a spacer and is formed with a single aperture.

Preferably, the bacterial filter assembly includes a bundle of mutually spaced hollow fibers.

There is further provided in accordance with yet another preferred embodiment of the present invention a bacteria-retaining medical valve including a housing defining a liquid inlet and a liquid outlet, a valve located within the housing downstream of the liquid inlet and a bacterial filter assembly located within the housing downstream of the valve and upstream of the liquid outlet, the bacterial filter assembly being formed of a plurality of hollow fibers and including hydrophilic bacterial impermeable passageways which are configured to permit liquid passage therethrough and prevent bacterial passage therethrough and air passage therethrough and hydrophobic bacterial impermeable passageways which are configured to permit air passage therethrough and prevent bacterial passage therethrough and liquid passage therethrough.

Preferably, at least an upstream part of the bacterial filter assembly reduces the pressure of liquid reaching at least a downstream part of the bacterial filter assembly and at least some of the hydrophobic bacterial impermeable passageways are located so as to prevent air buildup within the bacterial filter assembly at at least some of the hydrophilic bacterial impermeable passageways at which the liquid pressure is substantially reduced, which would otherwise block liquid flow therethrough.

There is even further provided in accordance with still another preferred embodiment of the present invention a bacteria-retaining medical valve including a housing defining a liquid inlet, a liquid outlet and an air vent aperture, a valve located within the housing downstream of the liquid inlet and a bacterial filter assembly located within the housing downstream of the valve and upstream of the liquid outlet, including at least one filter substrate and at least one spacer folded together to define a folded structure including hydrophilic bacterial impermeable passageways which are configured to permit liquid passage therethrough and prevent bacterial passage therethrough and air passage therethrough and hydrophobic bacterial impermeable passageways communicating with the air vent aperture, which are configured to permit air passage therethrough and prevent bacterial passage therethrough and liquid passage therethrough.

In accordance with a preferred embodiment of the present invention the bacterial filter assembly includes a folded bacterial filter element which, when folded, has multiple, non co-planar liquid and air passage surfaces. Preferably, the bacterial filter assembly includes a spiral wound filter element and spacer. Additionally or alternatively, the bacterial filter assembly includes an envelope formed of a filter substrate which encloses a spacer and is formed with a single aperture.

In accordance with a preferred embodiment of the present invention the bacterial filter assembly includes a bundle of mutually spaced hollow fibers.

### BRIEF DESCRIPTION OF THE DRAWINGS

The present invention will be understood and appreciated more fully from the following detailed description, taken in conjunction with the drawings in which:

FIGS. 1A & 1B are respective simplified exploded view and assembled view illustrations of a bacteria-retaining medical valve constructed and operative in accordance with a first embodiment of the present invention;

FIG. 2 is a detailed simplified side view sectional illustration of one embodiment of a bacterial filter assembly forming part of the bacteria-retaining medical valve of FIGS. 1A & 1B;

FIG. 3 is a detailed simplified, partially symbolic, side view sectional illustration of the operation of the bacterial filter assembly of FIG. 2;

FIG. 4 is a detailed simplified side view sectional illustration of another embodiment of a bacterial filter assembly suitable for use in the bacteria-retaining medical valve of FIGS. 1A & 1B;

FIG. 5 is a detailed simplified, partially symbolic, side view sectional illustration of the operation of the bacterial filter assembly of FIG. 4;

FIG. 6 is a detailed simplified side view sectional illustration of yet another embodiment of a bacterial filter assembly suitable for use in the bacteria-retaining medical valve of FIGS. 1A & 1B;

FIG. 7 is a detailed simplified side view sectional illustration of still another embodiment of a bacterial filter assembly suitable for use in the bacteria-retaining medical valve of FIGS. 1A & 1B;

FIG. 8 is a detailed simplified, partially symbolic, side view sectional illustration of the operation of the bacterial filter assemblies of FIGS. 6 & 7;

FIGS. 9A & 9B are respective simplified exploded view and assembled view illustrations of a bacteria-retaining medical valve constructed and operative in accordance with a further embodiment of the present invention;

FIG. 10 is a detailed simplified side view sectional illustration of one embodiment of a bacterial filter assembly forming part of the bacteria-retaining medical valve of FIGS. 9A & 9B;

FIG. 11 is a detailed simplified, partially symbolic, side view sectional illustration of the operation of the bacterial filter assembly of FIG. 10;

FIGS. 12A & 12B are respective simplified exploded view and assembled view illustrations of a bacteria-retaining medical valve constructed and operative in accordance with yet another embodiment of the present invention;

FIG. 13 is a detailed simplified side view sectional illustration of one embodiment of a bacterial filter assembly forming part of the bacteria-retaining medical valve of FIGS. 12A & 12B; and

FIG. 14 is a detailed simplified, partially symbolic, side view sectional illustration of the operation of the bacterial filter assembly of FIG. 13.

### DETAILED DESCRIPTION OF PREFERRED EMBODIMENTS

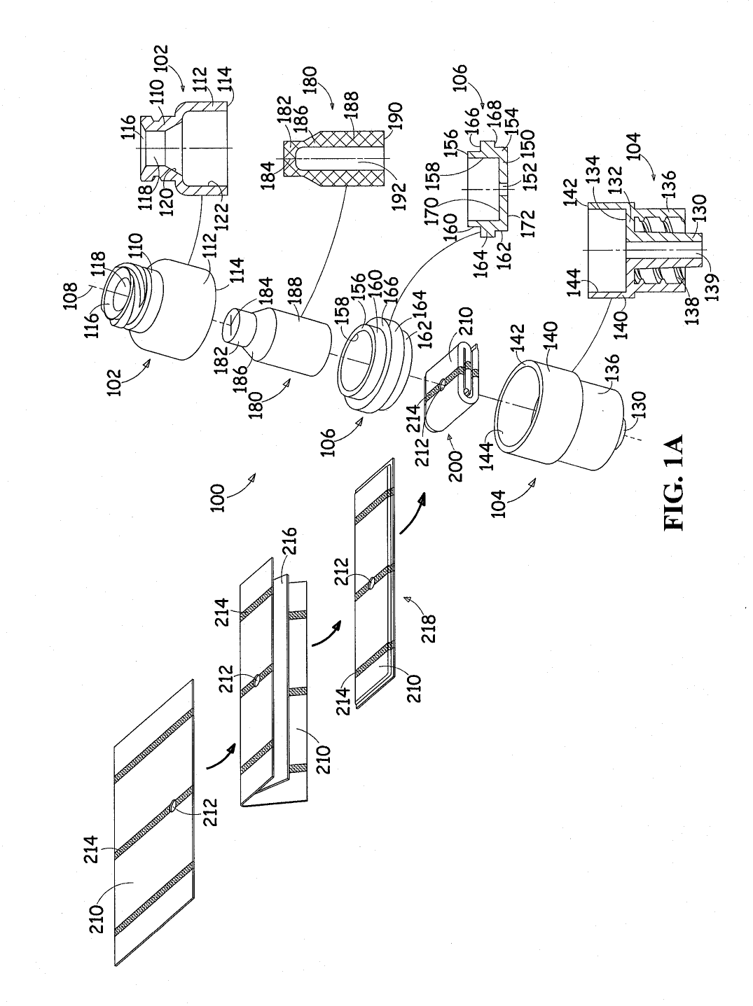

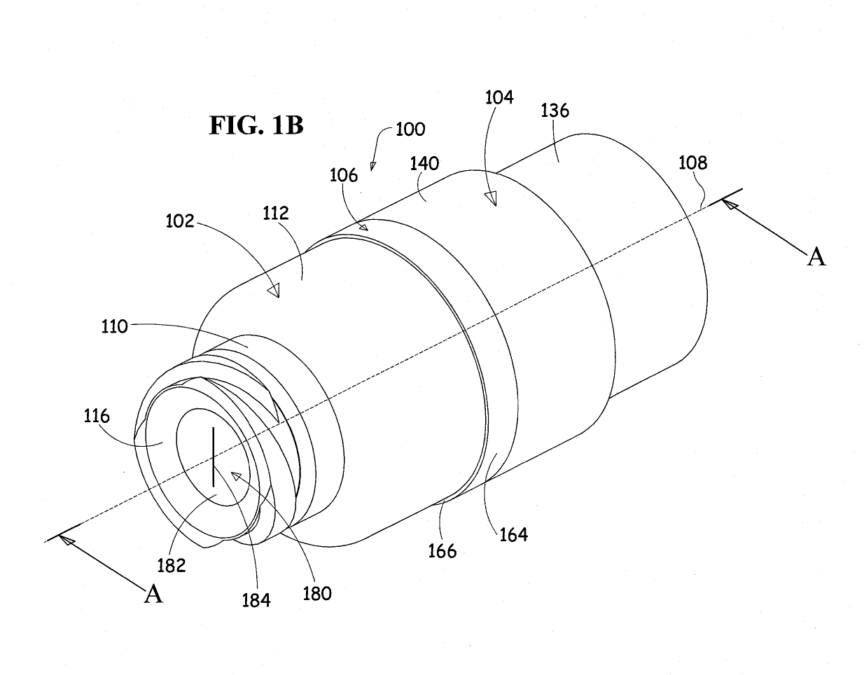

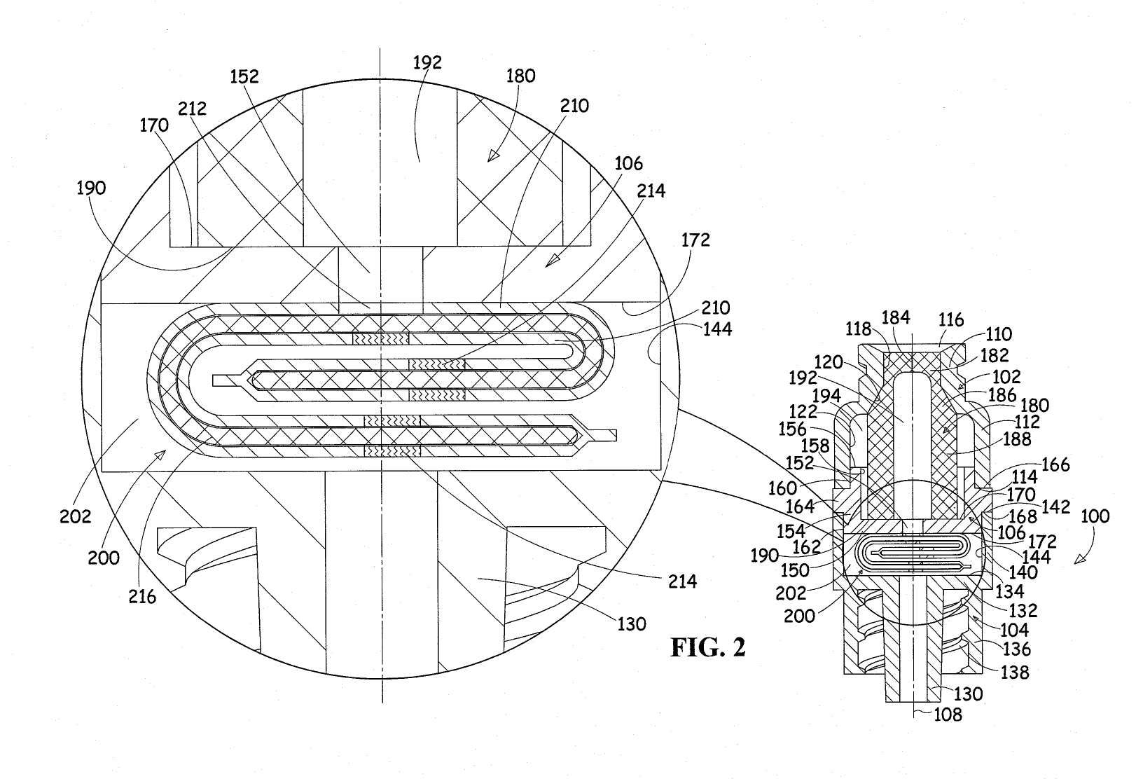

Reference is now made to FIGS. 1A & 1B, which are respective simplified exploded view and assembled view illustrations of a bacteria-retaining medical valve constructed and operative in accordance with a first embodiment of the present invention, and to FIG. 2, which is a detailed simplified side view sectional illustration of one embodiment of a bacterial filter assembly forming part of the bacteria-retaining medical valve of FIGS. 1A & 1B.

As seen in FIGS. 1A & 1B and in FIG. 2, there is provided a bacteria-retaining medical valve 100 having a housing assembly including a forward housing portion 102, a rearward housing portion 104 and a support element 106 disposed therebetween along an axis 108.

The forward housing portion 102 preferably includes a forward, outwardly threaded, generally hollow cylindrical wall portion 110 and a rearward forwardly rounded, generally hollow cylindrical wall portion 112 having a rearward edge 114. Forward generally hollow cylindrical wall portion 110 defines a tapered axial opening 116 which leads into a generally cylindrical bore 118, thence to a rearwardly tapered portion 120 and a broadened, forwardly curved portion 122. Part of rearwardly tapered portion 120 and forwardly curved portion 122 lies interiorly of rearward forwardly rounded, generally hollow cylindrical wall portion 112.

The rearward housing portion 104 defines a central axial male luer portion 130, which terminates in a flange 132, having a forwardly facing surface 134 and from which a rearward outer cylindrical wall portion 136 extends rearwardly. Rearward outer cylindrical wall portion 136 is spaced from and overlies part of the male luer portion 130 and is formed with interior luer lock threading 138 on an inner cylindrical surface thereof and a central bore 139. Extending forwardly from flange 132 is a forward outer cylindrical wall 140 having a forward edge 142 and defining an interior cylindrical wall surface 144.

The support element 106 preferably comprises an apertured base 150 having a central axial aperture 152. Extending from aperture base 150 is a circumferential wall portion 154 having a forward edge 156 and defining an interior facing cylindrical wall surface 158. Circumferential wall portion 154 defines a forward outwardly facing cylindrical wall surface 160, a rearward outwardly facing cylindrical wall surface 162 and a flange 164 therebetween, which defines respective forward and rearward facing ring surfaces 166 and 168. Base 150 defines a forwardly facing surface 170 and a rearwardly facing surface 172.

A valve 180, such as a Cat. Number 245204024, commercially available from Halkey Roberts of St. Petersburg, Fla., USA, or a CLAVE® valve, commercially available from ICU Medical of San Clemente, Calif., USA, here shown a Halkey Roberts valve, including a forward cylindrical portion 182 having an axial slit 184, a rearwardly outwardly tapered portion 186 and a cylindrical body portion 188, having a rearwardly facing surface 190, is preferably located within forward housing portion 102 and support element 106. Forward cylindrical portion 182 of valve 180 seats in generally cylindrical bore 118 of forward housing portion 102 and rearwardly outwardly tapered portion 186 of valve 180 seats in rearwardly tapered portion 120 of the forward housing portion 102. Valve 180 defines an axial fluid flow passageway 192 therewithin communicating with slit 184.

Cylindrical body portion 188 of valve 180 is located within a volume 194 (as shown in FIG. 2) defined by the interior of forwardly curved portion 122 of forward housing portion 102 and by the interior facing cylindrical wall surface 158 of support element 106. It is seen that preferably rearwardly facing surface 190 of valve 180 seats against forwardly facing surface 170 of support element 106, when the forward housing portion 102 is sealed to the support element 106, as by ultrasonic sealing, with rearwardly facing surface 114 of forward housing portion 102 abutting forwardly facing ring surface 166 of support element 106.

A bacterial filter assembly 200 is preferably located in a volume 202 (as shown in FIG. 2) defined by interior cylindrical wall surface 144 and forwardly facing surface 134 of rearward housing portion 104 and rearwardly facing surface 172 of base 150 of support element 106, when rearward housing portion 104 and support element 106 are sealed together, as by ultrasonic sealing, with forwardly facing edge 142 of rearward housing portion 104 abutting ring surface 168 of support element 106.

In accordance with one embodiment of the present invention, as seen in FIG. 1A, bacterial filter assembly 200 is preferably formed by providing a filter substrate 210, such as a SUPOR® membrane, commercially available from Pall Corporation of Port Washington, N.Y., USA, and aperturing it to define an entry passage 212 and forming thereon a plurality of hydrophobic regions 214, preferably by applying thereto a hydrophobic material, such as a siliconizing agent, for example MDX4®, commercially available from Dow Corning of Midland, Mich., USA. Following suitable curing, the filter substrate 210 is preferably folded, as shown in FIG. 1A, over a spacer sheet 216, such as a non-woven or netting sheet, for example a NALTEX® sheet, commercially available from Delstar Technologies of Middletown, Del., USA. Preferably, the folded filter substrate 210, surrounding the spacer sheet 216, is circumferentially sealed, as by ultrasonic or RF sealing, for example, to provide a precursor filter assembly 218. The precursor filter assembly 218 is preferably folded as shown so as to have entry passage defining aperture 212 centered on a top facing surface thereof, as shown, to define filter assembly 200.

As seen in FIG. 2, the filter assembly 200 is preferably adhered to rearward facing surface 172 of support element 106, as by ultrasonic or RF sealing, and arranged such that entry passage defining aperture 212 is aligned with aperture 152 of support element 106. It is further seen that aperture 152 of support element 106 communicates with fluid passage 192 of valve 180 and that the downstream side of filter assembly 200 communicates with the interior of volume 202, which in turn communicates with a fluid passageway defined by the interior of male luer 130.

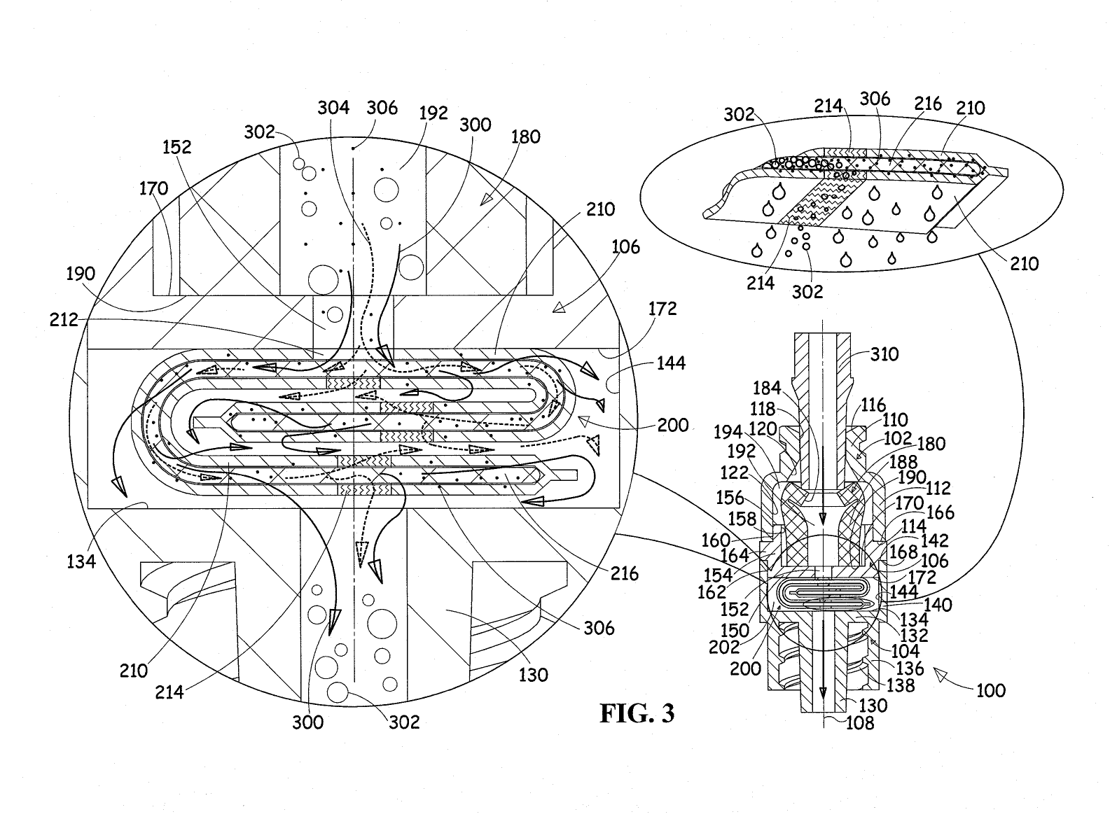

Reference is now made to FIG. 3, which is a detailed simplified, partially symbolic, side view sectional illustration of the operation of the bacterial filter assembly of FIG. 2. In FIG. 3, for ease of understanding, liquid flows are shown by solid arrows, here designated by reference numeral 300, air bubbles are designated by reference numeral 302, flows of air bubbles are shown by dashed arrows, here designated by reference numeral 304, and bacteria are symbolized by small dots, here designated by reference numeral 306.

When a male luer 310 is inserted at the forward end of the bacteria-retaining medical valve 100, engaging opening 116 and bore 118 of forward housing portion 102, and thereby opening slit 184, fluid, such as an infusion solution in which both air bubbles 302 and bacteria 306 may be present, flows through the male luer 310 and passageway 192 of valve 180 via aperture 152 and entry passage 212 to the interior of bacterial filter assembly 200. Inside bacterial filter assembly 200, the fluid passages along and through spacer 216 and the bacteria 306 are retained in the filter substrate 210. Air bubbles 302 pass through the hydrophobic regions 214 of the filter substrate 210 which define hydrophobic bacterial impermeable passageways and liquid passes through the remaining regions of the filter substrate 210, which are hydrophilic and which define hydrophilic bacterial impermeable passageways.

It is appreciated that in an embodiment of the present invention, at least an upstream part of bacterial filter assembly 200 reduces the pressure of liquid reaching at least a downstream part of the bacterial filter assembly 200. This pressure reduction is produced, for example, by the passage of liquid inside bacterial filter assembly 200, along the fluid passages along and through spacer 216.

Thus it is understood that at least one pressure reducing passageway substantially reduces the pressure of liquid reaching at least some of the hydrophilic bacterial impermeable passageways downstream thereof.

It is a particular feature of the present invention that at least some of the hydrophobic bacterial impermeable passageways are located so as to prevent air buildup within the bacterial filter assembly 200 at those hydrophilic bacterial impermeable passageways at which the liquid pressure is substantially reduced, which would otherwise block liquid flow therethrough.

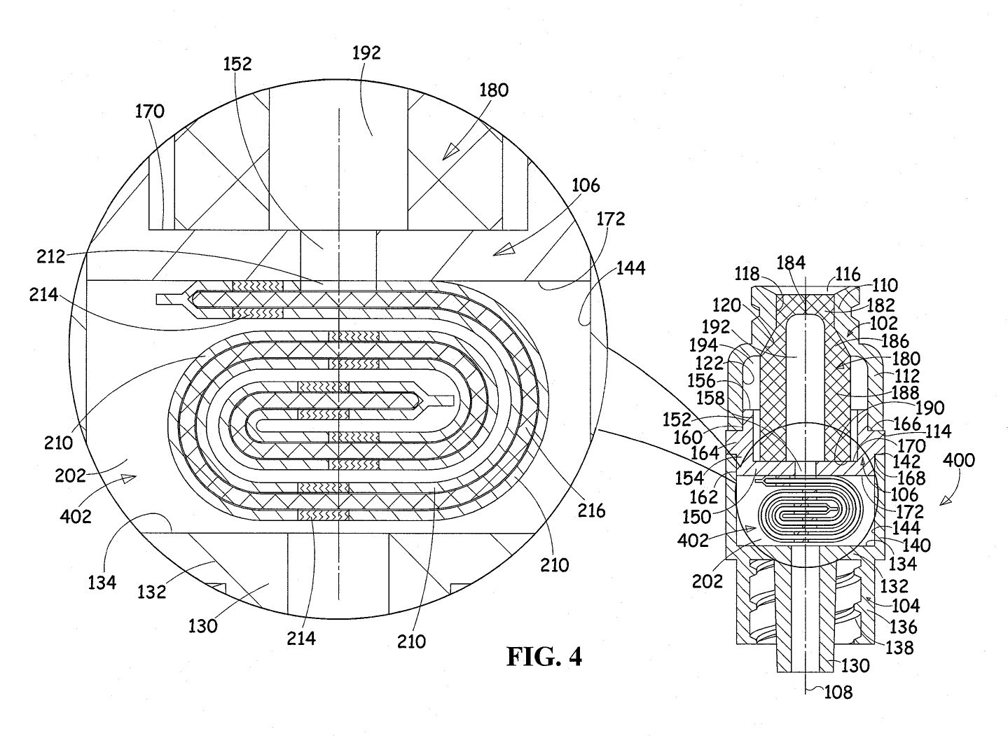

Reference is now made to FIG. 4, which is a detailed simplified side view sectional illustration of another embodiment of a bacterial filter assembly suitable for use in a bacteria-retaining medical valve 400 which is generally similar to the bacteria-retaining medical valve 100 of FIGS. 1A & 1B.

Elements of the bacteria-retaining medical valve 400 which are identical to those of bacteria-retaining medical valve 100 are designated by identical reference numerals.

In the embodiment of FIGS. 4 & 5, the precursor filter assembly 218 (FIG. 1A) is preferably spiral wound, as shown in FIGS. 4 & 5, so as to have entry passage defining aperture 212 (FIGS. 1A & 2) centered on a top facing surface thereof, as shown to define filter assembly 200 (FIGS. 1A & 2) in a spiral wound configuration, which is designated by reference numeral 402.

As seen in FIG. 4, the filter assembly 402 is preferably adhered to rearward facing surface 172 (FIGS. 1A & 2) of support element 106 (FIGS. 1A & 2), as by ultrasonic or RF sealing, and arranged such that entry passage defining aperture 212 (FIGS. 1A & 2) is aligned with aperture 152 (FIGS. 1A & 2) of support element 106 (FIGS. 1A & 2). It is further seen that aperture 152 (FIGS. 1A & 2) of support element 106 (FIGS. 1A & 2) communicates with fluid passage 192 (FIGS. 1A & 2) of valve 180 (FIGS. 1A & 2) and that the downstream side of filter assembly 402 communicates with the interior of volume 202 (FIGS. 1A & 2) which in turn communicates with a fluid passageway defined by the interior of male luer 130.

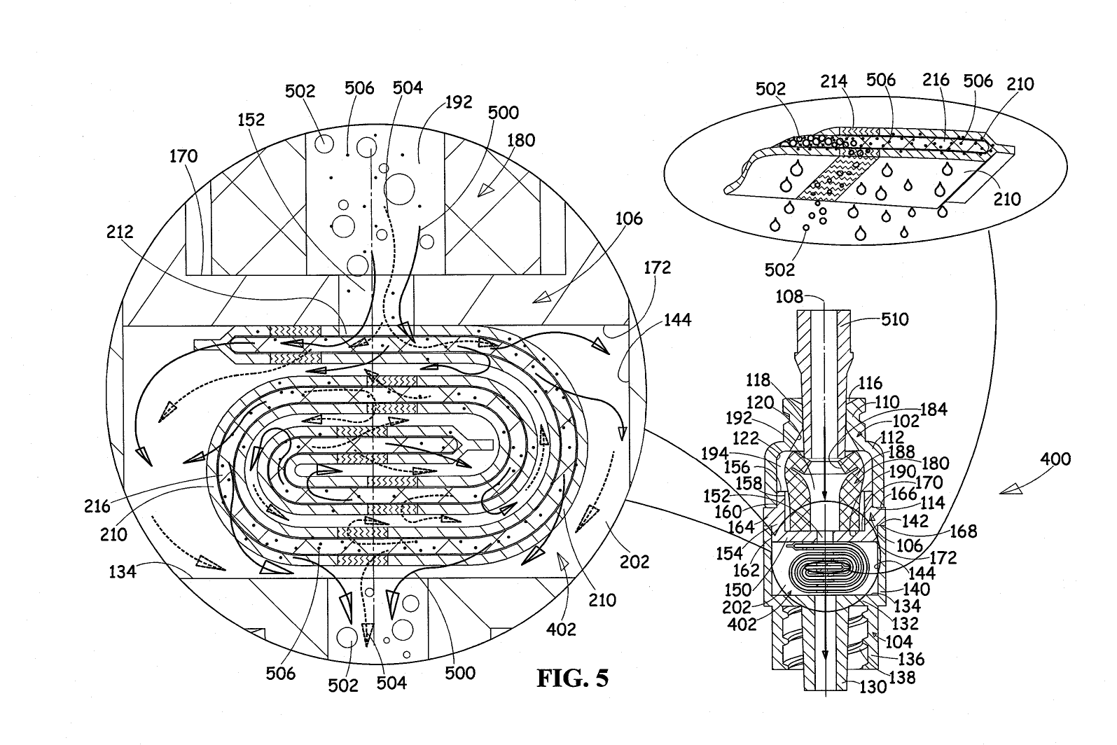

Reference is now made to FIG. 5, which is a detailed simplified, partially symbolic, side view sectional illustration of the operation of the bacterial filter assembly of FIG. 4. In FIG. 5, for ease of understanding, liquid flows are shown by solid arrows, here designated by reference numeral 500, air bubbles are designated by reference numeral 502, flows of air bubbles are shown by dashed arrows, here designated by reference numeral 504 and bacteria are symbolized by small dots, here designated by reference numeral 506.

When a male luer 510 is inserted at the forward end of the bacteria-retaining medical valve 400, engaging opening 116 and bore 118 of forward housing portion 102, and thereby opening slit 184, fluid, such as an infusion solution in which both air bubbles 502 and bacteria 506 may be present, flows through the male luer 510 and fluid passage 192 of valve 180 via aperture 152 and entry passage 212 to the interior of bacterial filter assembly 402. Inside bacterial filter assembly 402, the fluid passes along and through spacer 216 and the bacteria 506 are retained in the filter substrate 210. Air bubbles 502 pass through the hydrophobic regions 214 of the filter substrate 210 which define hydrophobic bacterial impermeable passageways and liquid passes through the remaining regions of the filter substrate 210, which are hydrophilic and which define hydrophilic bacterial impermeable passageways.

It is appreciated that in this embodiment of the present invention, at least an upstream part of bacterial filter assembly 402 reduces the pressure of liquid reaching at least a downstream part of the bacterial filter assembly 402.

Thus it is understood that at least one pressure reducing passageway substantially reduces the pressure of liquid reaching at least some of the hydrophilic bacterial impermeable passageways downstream thereof.

It is a particular feature of the present invention that at least some of the hydrophobic bacterial impermeable passageways are located so as to prevent air buildup within the bacterial filter assembly 402 at those hydrophilic bacterial impermeable passageways at which the liquid pressure is substantially reduced, which would otherwise block liquid flow therethrough.

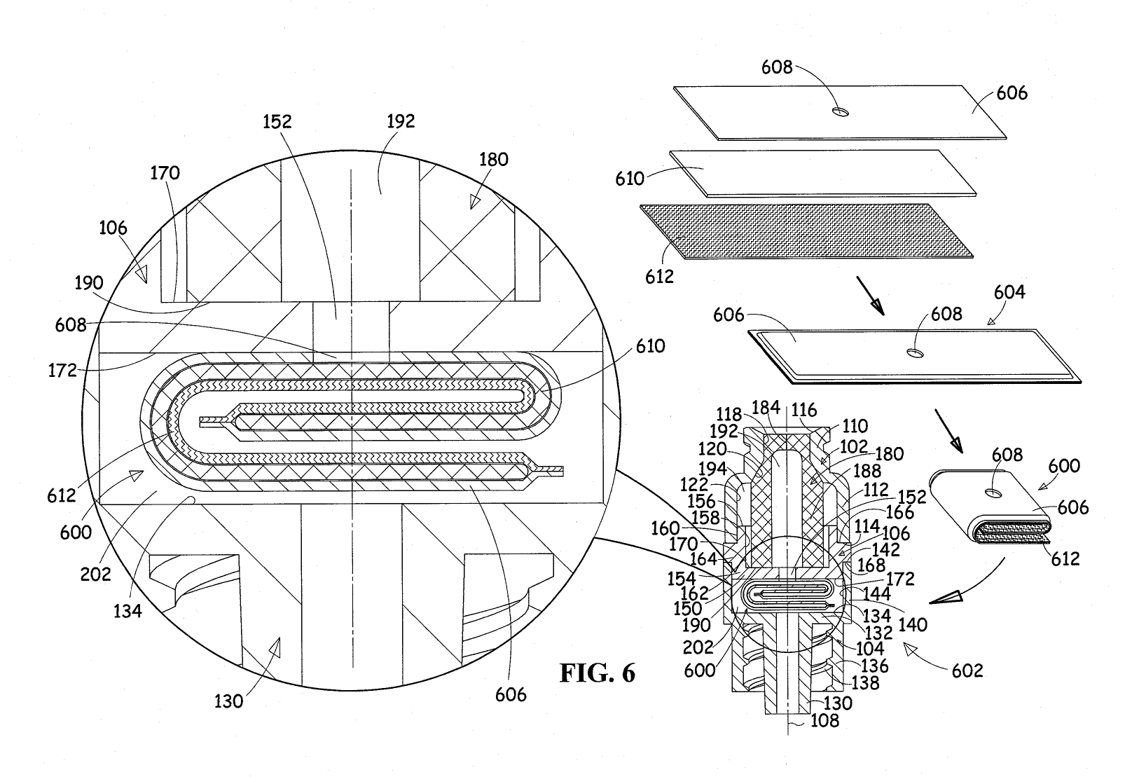

Reference is now made to FIG. 6, which is a detailed simplified side view sectional illustration of yet another embodiment of a bacterial filter assembly 600 suitable for use in a bacteria-retaining medical valve 602 which is generally similar to the bacteria-retaining medical valve 100 of FIGS. 1A & 1B. Elements of the bacteria-retaining medical valve 602 which are identical to those of bacteria-retaining medical valve 100 are designated by identical reference numerals.

In the embodiment of FIG. 6, a precursor filter assembly 604 is formed from a hydrophilic filter substrate 606 having an entry passage defining aperture 608 formed therein, such as a SUPOR® 200 membrane, commercially available from Pall Corporation of Port Washington, N.Y., USA, a spacer sheet 610, such as a non-woven or netting sheet, for example a NALTEX® sheet, commercially available from Delstar Technologies of Middletown, Del., USA and a hydrophobic filter substrate 612, such as a SUPOR® 450R membrane, commercially available from Pall Corporation of Port Washington, N.Y., USA.

The hydrophilic filter substrate 606, the spacer sheet 610 and the hydrophobic filter substrate 612 are circumferentially sealed together, as by ultrasonic or heat sealing, for example, to provide a precursor filter assembly 604.

The precursor filter assembly 604 is preferably folded as shown so as to have entry passage defining aperture 608 centered on a top facing surface thereof, as shown, to define filter assembly 600.

The filter assembly 600 is preferably adhered to rearward facing surface 172 (FIGS. 1A & 2) of support element 106 (FIGS. 1A & 2), as by ultrasonic or RF sealing, and arranged such that entry passage defining aperture 608 is aligned with aperture 152 (FIGS. 1A & 2) of support element 106 (FIGS. 1A & 2). It is further seen that aperture 152 (FIGS. 1A & 2) of support element 106 (FIGS. 1A & 2) communicates with fluid passage 192 (FIGS. 1A & 2) of valve 180 (FIGS. 1A & 2) and that the downstream side of filter assembly 600 communicates with the interior of volume 202 (FIGS. 1A & 2), which in turn communicates with a fluid passageway defined by the interior of male luer 130 (FIGS. 1A & 2).

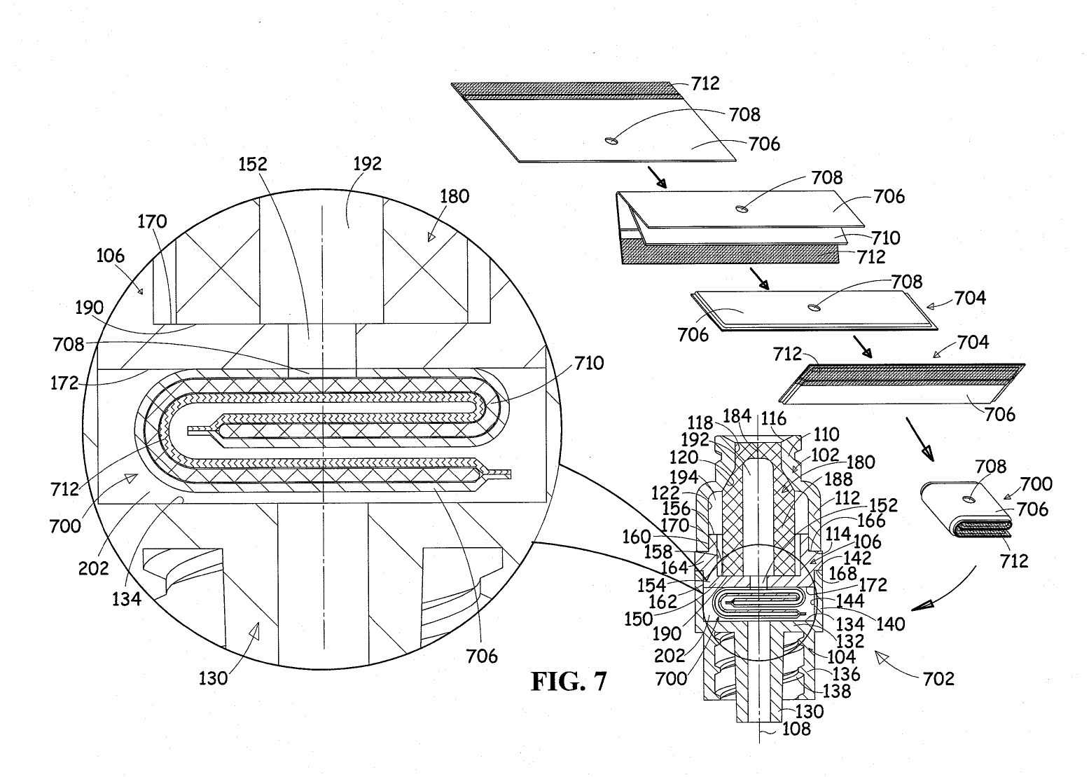

Reference is now made to FIG. 7, which is a detailed simplified side view sectional illustration of still another embodiment of a bacterial filter assembly 700 suitable for use in a bacteria-retaining medical valve 702 which is generally similar to the bacteria-retaining medical valve 100 of FIGS. 1A & 1B. Elements of the bacteria-retaining medical valve 702 which are identical to those of bacteria-retaining medical valve 100 are designated by identical reference numerals.

In the embodiment of FIG. 7, a precursor filter assembly 704 is formed from a hydrophilic filter substrate 706 having an entry passage defining aperture 708 formed therein, such as a SUPOR® 200 membrane, commercially available from Pall Corporation of Port Washington, N.Y., USA, a spacer 710, such as a non-woven or netting sheet, for example a NALTEX® sheet, commercially available from Delstar Technologies of Middletown, Del., USA and a hydrophobic filter substrate 712, such as a SUPOR® 450R membrane, commercially available from Pall Corporation of Port Washington, N.Y., USA. The hydrophilic filter substrate 706 is sealed to the hydrophobic filter substrate 712 edge over edge and they are folded over the spacer sheet 710 as shown in FIG. 7. Preferably, the folded filter substrate 706 and hydrophobic filter substrate 712, surrounding the spacer sheet 710, is circumferentially sealed, as by ultrasonic or RF sealing, for example, to provide a precursor filter assembly 704.

The precursor filter assembly 704 is preferably folded as shown so as to have entry passage defining aperture 708 centered on a top facing surface thereof, as shown, to define filter assembly 700.

The filter assembly 700 is preferably adhered to rearward facing surface 172 (FIGS. 1A & 2) of support element 106 (FIGS. 1A & 2), as by ultrasonic or RF sealing, and arranged such that entry passage defining aperture 708 is aligned with aperture 152 (FIGS. 1A & 2) of support element 106 (FIGS. 1A & 2). It is further seen that aperture 152 (FIGS. 1A & 2) of support element 106 (FIGS. 1A & 2) communicates with fluid passage 192 (FIGS. 1A & 2) of valve 180 (FIGS. 1A & 2) and that the downstream side of filter assembly 700 communicates with the interior of volume 202 (FIGS. 1A & 2), which in turn communicates with a fluid passageway defined by the interior of male luer 130 (FIGS. 1A & 2).

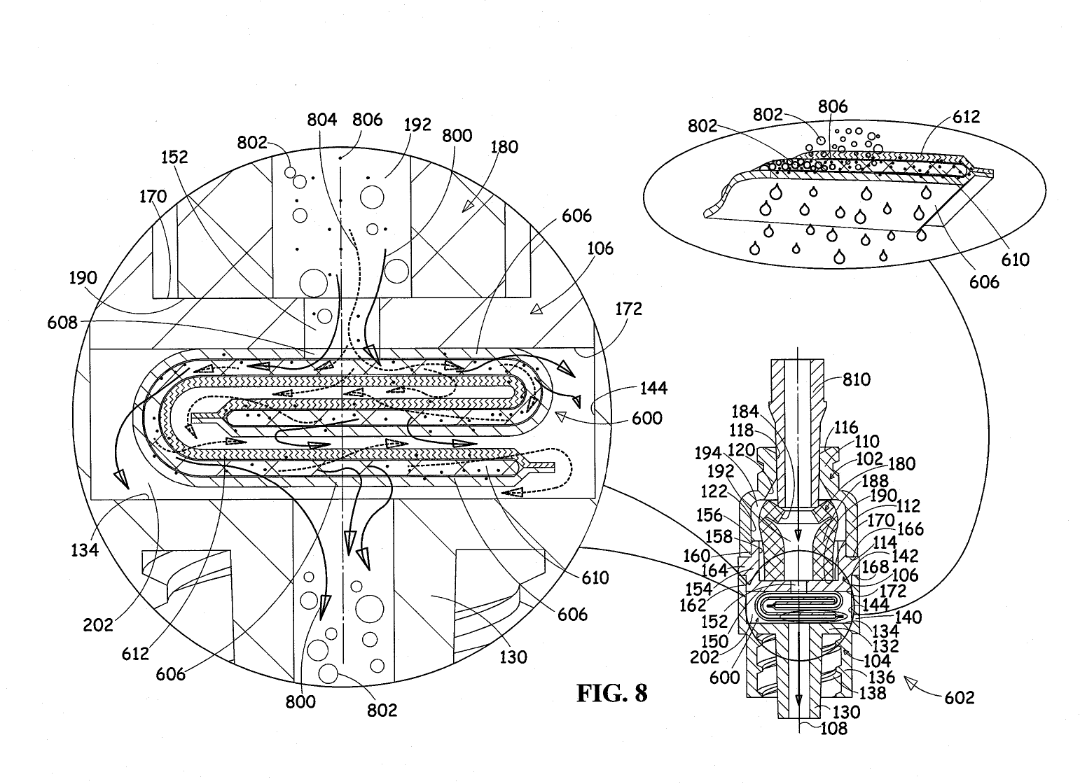

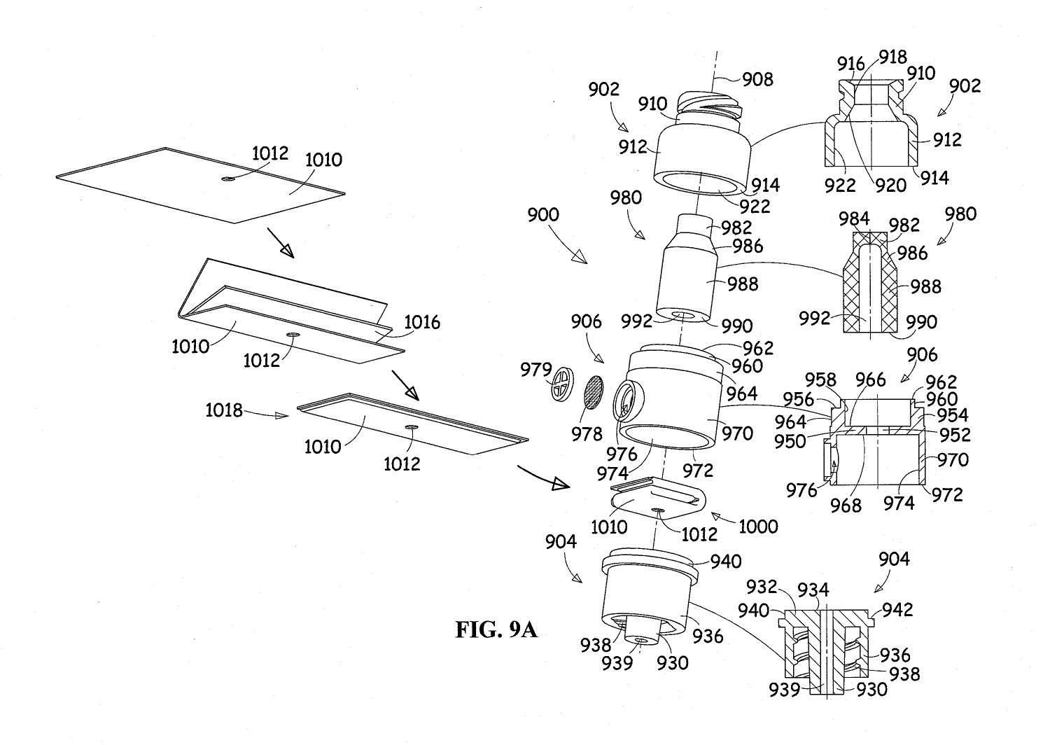

Reference is now made to FIG. 8, which is a detailed simplified, partially symbolic, side view sectional illustration of the operation of the bacterial filter assemblies of FIGS. 6 & 7, which look the same in FIG. 8. For the sake of simplicity and clarity, in the description of FIG. 8, which follows, the reference numerals of FIG. 6 are employed, it being understood that the description of FIG. 8 which follows is equally applicable to the embodiment of FIG. 7.

In FIG. 8, for ease of understanding, liquid flows are shown by solid arrows, here designated by reference numeral 800, air bubbles are designated by reference numeral 802, flows of air bubbles are shown by dashed arrows, here designated by reference numeral 804, and bacteria are symbolized by small dots, here designated by reference numeral 806.

When a male luer 810 is inserted at the forward end of the bacteria-retaining medical valve 602, engaging opening 116 (FIGS. 1A & 2) and bore 118 (FIGS. 1A & 2) of forward housing portion 102 (FIGS. 1A & 2), and thereby opening slit 184 (FIGS. 1A & 2), fluid, such as an infusion solution in which both air bubbles 802 and bacteria 806 may be present, flows through the male luer 810 and passageway 192 (FIGS. 1A & 2) of valve 180 (FIGS. 1A & 2) via aperture 152 (FIGS. 1A & 2) and entry passage 608 to the interior of bacterial filter assembly 600. Inside bacterial filter assembly 600, the fluid passes along and through spacer sheet 610 and the bacteria 806 are retained in the filter substrate 606. Air bubbles 802 pass through the hydrophobic substrate 612 of the bacterial filter assembly 600, which define hydrophobic bacterial impermeable passageways, and liquid passes through the remaining regions of the filter substrate 606, which are hydrophilic and which define hydrophilic bacterial impermeable passageways.

It is appreciated that in this embodiment of the present invention, at least an upstream part of bacterial filter assembly 600 reduces the pressure of liquid reaching at least a downstream part of the bacterial filter assembly 600.

Thus it is understood that at least one pressure reducing passageway substantially reduces the pressure of liquid reaching at least some of the hydrophilic bacterial impermeable passageways downstream thereof.

It is a particular feature of the present invention that at least some of the hydrophobic bacterial impermeable passageways are located so as to prevent air buildup within the bacterial filter assembly 600 at those hydrophilic bacterial impermeable passageways at which the liquid pressure is substantially reduced, which would otherwise block liquid flow therethrough.

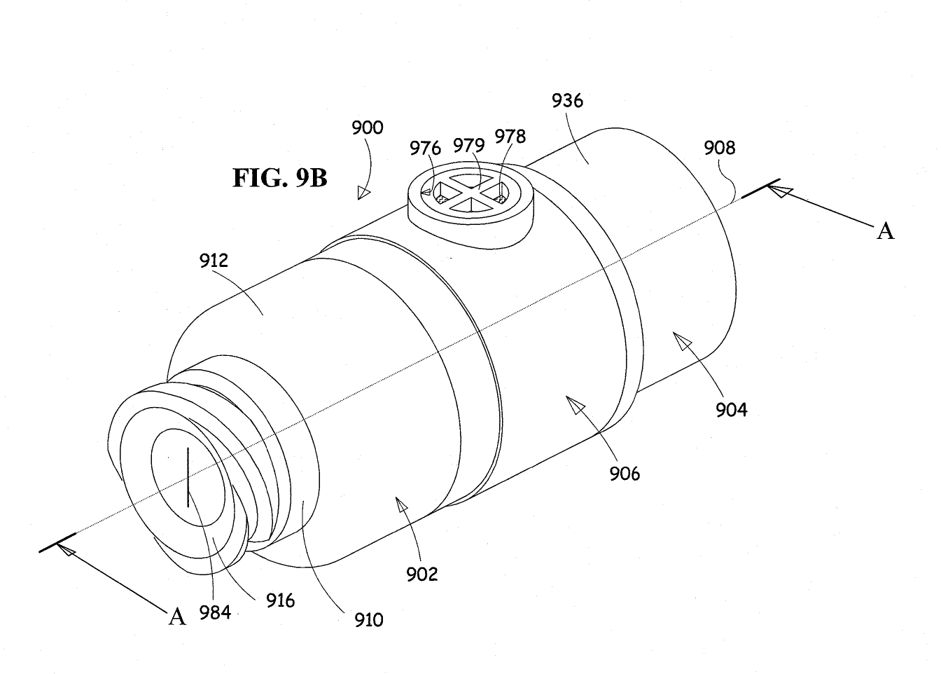

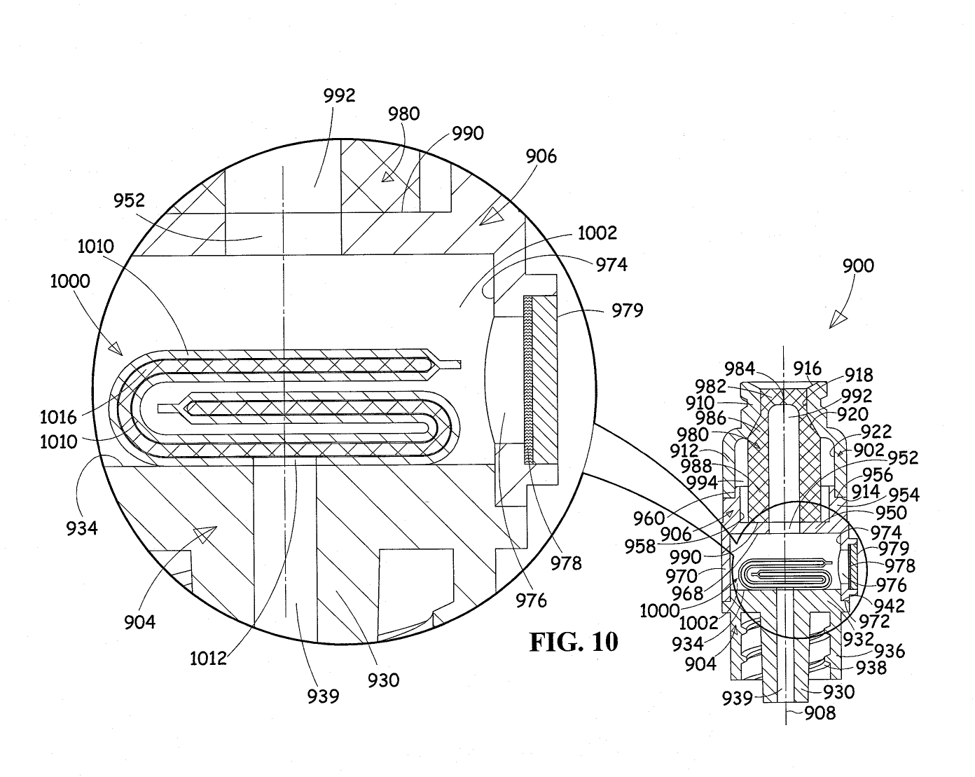

Reference is now made to FIGS. 9A & 9B, which are respective simplified exploded view and assembled view illustrations of a bacteria-retaining medical valve constructed and operative in accordance with further embodiment of the present invention, and to FIG. 10, which is a detailed simplified side view sectional illustration of one embodiment of a bacterial filter assembly forming part of the bacteria-retaining medical valve of FIGS. 9A & 9B.

As seen in FIGS. 9A & 9B and in FIG. 10, there is provided a bacteria retaining medical valve 900 having a housing assembly including a forward housing portion 902, a rearward housing portion 904 and a support element 906 disposed therebetween along an axis 908.

The forward housing portion 902 preferably includes a forward, outwardly threaded, generally hollow cylindrical wall portion 910 and a rearward forwardly rounded, generally hollow cylindrical wall portion 912 having a rearward edge 914. Forward generally hollow cylindrical wall portion 910 defines an tapered axial opening 916 which leads into a generally cylindrical bore 918, thence to a rearwardly tapered portion 920 and a broadened, forwardly curved portion 922. Part of rearwardly tapered portion 920 and forwardly curved portion 922 lies interiorly of rearward forwardly rounded, generally hollow cylindrical wall portion 912.

The rearward housing portion 904 defines a central axial male luer portion 930, which terminates in a flange 932, having a forwardly facing surface 934 and from which a rearward outer cylindrical wall portion 936 extends rearwardly. Rearward outer cylindrical wall portion 936 is spaced from and overlies part of the male luer portion 930 and is formed with interior luer lock threading 938 on an inner cylindrical surface thereof and a central bore 939. Extending rearwardly from flange 932 is a forward outer cylindrical wall 940 defining a forwardly facing outwardly protruding ring surface 942.

The support element 906 preferably comprises an apertured base 950 having a central axial aperture 952 from which extends a forwardly extending circumferential wall portion 954, having a forward edge 956 and defining an interior facing cylindrical wall surface 958. Circumferential wall portion 954 defines a forward outwardly facing cylindrical wall surface 960 which defines a forward facing ring surface 962 and a rearward outwardly facing cylindrical wall surface 964. Base 950 defines a forwardly facing surface 966 and a rearwardly facing surface 968. Extending rearwardly from base 950 is a rearwardly extending circumferential wall portion 970 having a rearward edge 972 and defining an interior facing cylindrical wall surface 974 in which there is provided an air vent hole and socket 976, which accommodates an air vent filter 978, such as a SUPOR® 450R membrane, commercially available from Pall Corporation of Port Washington, N.Y., USA, and an air vent cover 979.

A valve 980, such as a Cat. Number 245204024, commercially available from Halkey Roberts of St. Petersburg, Fla., USA, or a CLAVE® valve, commercially available from ICU Medical of San Clemente, Calif., USA, here shown a Halkey Roberts valve, including a forward cylindrical portion 982 having an axial slit 984, a rearwardly outwardly tapered portion 986 and a cylindrical body portion 988, having a rearwardly facing surface 990, is preferably located within forward housing portion 902 and support element 906. Forward cylindrical portion 982 of valve 980 seats in generally cylindrical bore 918 of forward housing portion 902 and rearwardly outwardly tapered portion 986 of valve 980 seats in rearwardly tapered portion 920 of the forward housing portion 902. Valve 980 defines an axial fluid flow passageway 992 therewithin communicating with slit 984.

The cylindrical body portion 988 of valve 980 is located within a volume 994 (as seen in FIG. 10) defined by the interior of forwardly curved portion 922 of forward housing portion 902 and by the interior facing cylindrical wall surface 958 of support element 906. It is seen that preferably rearwardly facing surface 990 of valve 980 seats against forwardly facing surface 966, when the forward housing portion 902 is sealed to the support element 906, as by ultrasonic sealing, with rearwardly facing surface 914 of forward housing portion 902 abutting forward edge 956 of support element 906.

A bacterial filter assembly 1000 is preferably located in a volume 1002 (as shown in FIG. 10) defined by forwardly facing surface 934 of rearward housing portion 904, interior facing cylindrical wall surface 974 of support element 906 and rearwardly facing surface 968 of base 950 of support element 906, when rearward housing portion 904 and support element 906 are sealed together, as by ultrasonic sealing, with ring surface 942 of rearward housing portion 904 abutting rearward edge 972 of support element 906.

In accordance with one embodiment of the present invention, as seen in FIG. 9A, bacterial filter assembly 1000 is preferably formed by providing a filter substrate 1010, such as a SUPOR® membrane, commercially available from Pall Corporation of Port Washington, N.Y., USA and aperturing it to define an egress passage 1012. The filter substrate 1010 is preferably folded, as shown in FIG. 9A, over a spacer sheet 1016, such as a non-woven or netting sheet, for example a NALTEX® sheet, commercially available from Delstar Technologies of Middletown, Del., USA. Preferably the folded filter substrate 1010, surrounding the spacer sheet 1016, is circumferentially sealed, as by ultrasonic or RF sealing, for example to provide a precursor filter assembly 1018. The precursor filter assembly 1018 is preferably folded as shown so as to have egress passage defining aperture 1012 centered on a bottom facing surface thereof, as shown, to define filter assembly 1000.

As seen in FIG. 10, the filter assembly 1000 is preferably adhered to forwardly facing surface 934 of rearward housing portion 904, as by ultrasonic or RF sealing, and arranged such that egress passage defining aperture 1012 is aligned with bore 939 of male luer 930. It is further seen that aperture 952 of support element 906 communicates with fluid flow passageway 992 of valve 980 and that the upstream side of filter assembly 1000 communicates with the interior of volume 1002, which, as noted above, is vented by an air vent provided in support element 906.

The downstream side of filter assembly 1000 communicates with a fluid passageway defined by bore 939 of male luer 930.

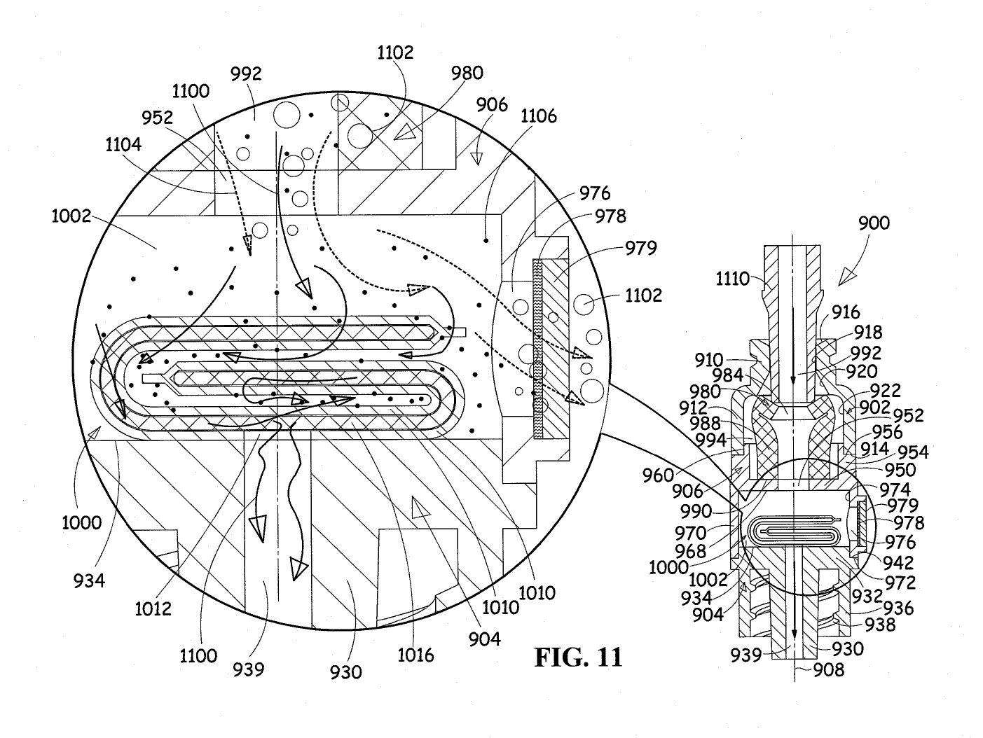

Reference is now made to FIG. 11, which is a detailed simplified, partially symbolic, side view sectional illustration of the operation of the bacterial filter assembly of FIG. 10. In FIG. 11, for ease of understanding, liquid flows are shown by solid arrows, here designated by reference numeral 1100, air bubbles are designated by reference numeral 1102, flows of air bubbles are shown by dashed arrows, here designated by reference numeral 1104, and bacteria are symbolized by small dots, here designated by reference numeral 1106.

When a male luer 1110 is inserted at the forward end of the bacteria-retaining medical valve 900, engaging opening 916 and bore 918 of forward housing portion 902, and thereby opening slit 984, fluid, such as an infusion solution in which both air bubbles 1102 and bacteria 1106 may be present, flows through the male luer 1110 and passageway 992 of valve 980 via aperture 952 to the interior of volume 1002. The air bubbles 1102 are vented through the air vent at vent hole 976 via air vent filter 978.

The remaining fluid passes through the filter substrate 1010 to the interior of bacterial filter assembly 1000. Inside bacterial filter assembly 1000, the fluid passes along and through spacer 1016 and the bacteria 1106 are retained in the filter substrate 1010.

It is thus appreciated that in this embodiment the bacterial filter assembly 1000 and the air vent filter 978 together provide a bacterial filter assembly including hydrophilic bacterial impermeable passageways which are configured to permit liquid passage therethrough and prevent bacterial passage therethrough and air passage therethrough and hydrophobic bacterial impermeable passageways which are configured to permit air passage therethrough and prevent bacterial passage therethrough and liquid passage therethrough.

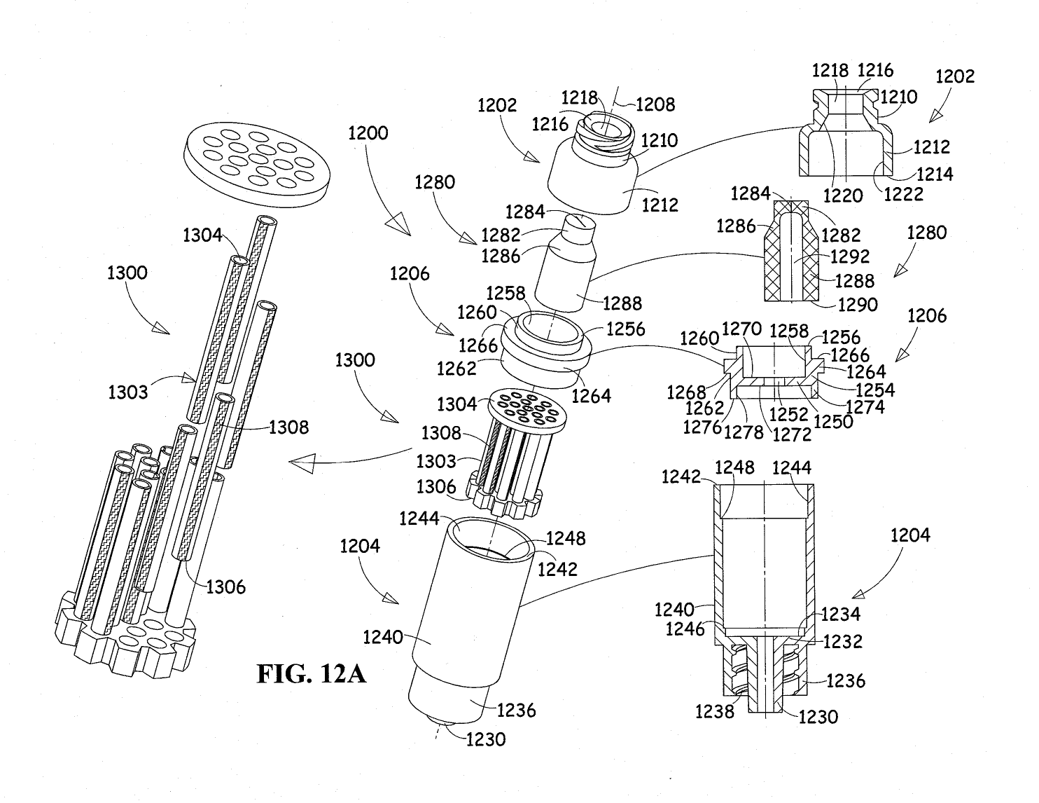

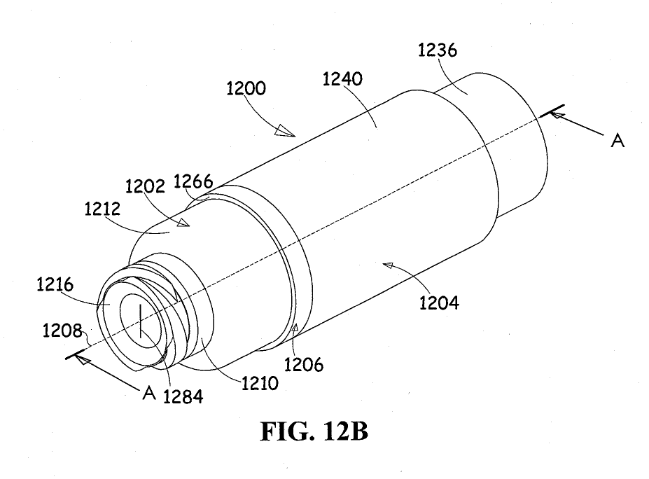

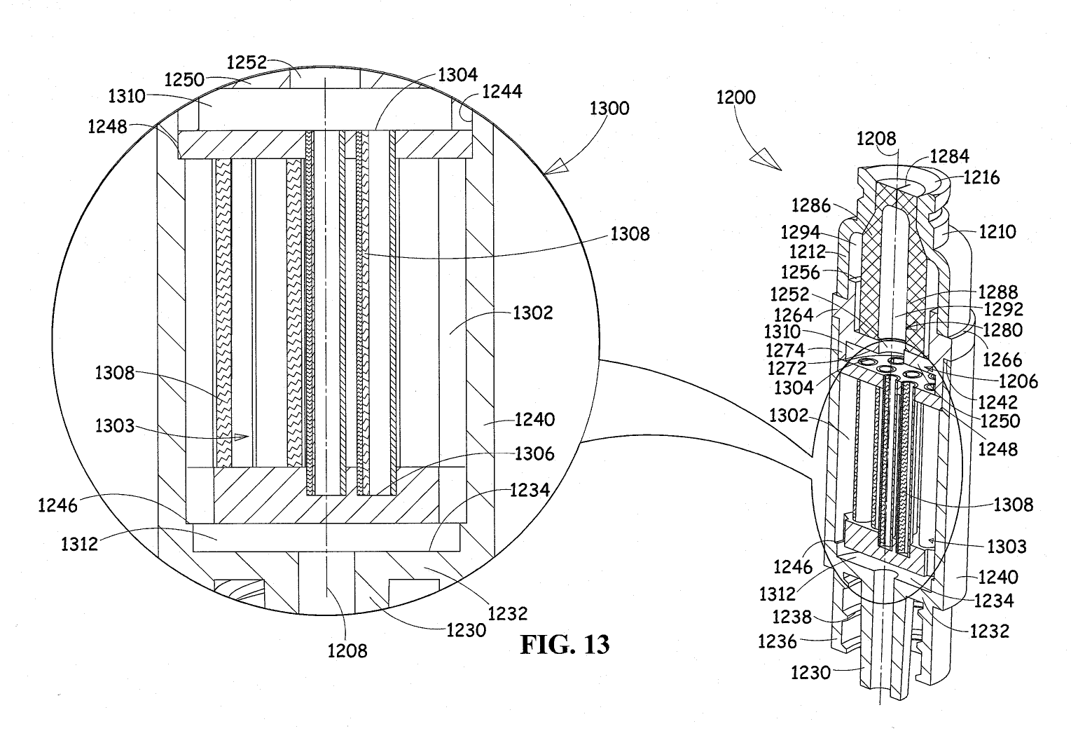

Reference is now made to FIGS. 12A & 12B, which are respective simplified exploded view and assembled view illustrations of a bacteria-retaining medical valve constructed and operative in accordance with yet a further embodiment of the present invention, and to FIG. 13, which is a detailed simplified side view sectional illustration of one embodiment of a bacterial filter assembly forming part of the bacteria-retaining medical valve of FIGS. 12A & 12B.

As seen in FIGS. 12A & 12B and in FIG. 13, there is provided a bacteria-retaining medical valve 1200 having a housing assembly including a forward housing portion 1202, a rearward housing portion 1204 and a support element 1206 disposed therebetween along an axis 1208.

The forward housing portion 1202 preferably includes a forward, outwardly threaded, generally hollow cylindrical wall portion 1210 and a rearward forwardly rounded, generally hollow cylindrical wall portion 1212 having a rearward edge 1214. Forward generally hollow cylindrical wall portion 1210 defines an tapered axial opening 1216 which leads into a generally cylindrical bore 1218, thence to a rearwardly tapered portion 1220 and a broadened, forwardly curved portion 1222. Part of rearwardly tapered portion 1220 and forwardly curved portion 1222 lies interiorly of rearward forwardly rounded, generally hollow cylindrical wall portion 1212.

The rearward housing portion 1204 defines a central axial male luer portion 1230, which terminates in a flange 1232, having a forwardly facing surface 1234 and from which a rearward outer cylindrical wall portion 1236 extends rearwardly. Rearward outer cylindrical wall portion 1236 is spaced from and overlies part of the male luer portion 1230 and is formed with interior luer lock threading 1238 on an inner cylindrical surface thereof. Extending forwardly from flange 1232 is a forward outer cylindrical wall 1240 having a forward edge 1242 and defining an interior generally cylindrical wall surface 1244. Interior generally cylindrical wall surface 1244 preferably defines a rearward circumferential shoulder 1246 and a forward circumferential shoulder 1248.

The support element 1206 preferably comprises an apertured base 1250 having a central axial aperture 1252 from which extends a forward circumferential wall portion 1254, having a forward edge 1256 and defining a forward interior facing cylindrical wall surface 1258. Circumferential wall portion 1254 defines a forward outwardly facing cylindrical wall surface 1260, a rearward outwardly facing cylindrical wall surface 1262 and a flange 1264 therebetween which defines respective forward and rearward facing ring surfaces 1266 and 1268. Base 1250 defines a forwardly facing surface 1270 and a rearwardly facing surface 1272. A rearward circumferential wall portion 1274 extends from base 1250. Wall portion 1274 has a rearward edge 1276 and defines a rearward interior facing cylindrical wall surface 1278. The outer surface of wall portion 1274 is a rearward continuation of rearward outwardly facing cylindrical wall surface 1262.

A valve 1280, such as a Cat. Number 245204024, commercially available from Halkey Roberts of St. Petersburg, Fla., USA, or a CLAVE® valve, commercially available from ICU Medical of San Clemente, Calif., USA, here shown a Halkey Roberts valve, including a forward cylindrical portion 1282 having an axial slit 1284, a rearwardly outwardly tapered portion 1286 and a cylindrical body portion 1288, having a rearwardly facing surface 1290, is preferably located within forward housing portion 1202 and support element 1206. Forward cylindrical portion 1282 of valve 1280 seats in generally cylindrical bore 1218 of forward housing portion 1202 and rearwardly outwardly tapered portion 1286 of valve 1280 seats in rearwardly tapered portion 1220 of the forward housing portion 1202. Valve 1280 defines an axial fluid flow passageway 1292 therewithin communicating with slit 1284.

The cylindrical body portion 1288 of valve 1280 is located within a volume 1294 defined by the interior of forwardly curved portion 1222 of forward housing portion 1202 and by the interior facing cylindrical wall surface 1258 of support element 1206. It is seen that preferably rearwardly facing surface 1290 of valve 1280 seats against forwardly facing surface 1270, when the forward housing portion 1202 is sealed to the support element 1206, as by ultrasonic sealing, with rearwardly facing surface 1214 of forward housing portion 1202 abutting forwardly facing ring surface 1266 of support element 1206.

A bacterial filter assembly 1300 is preferably located in an interior volume 1302 defined by interior cylindrical wall surface 1244 and forwardly facing surface 1234 of rearward housing portion and rearwardly facing surface 1272 of base 1250 of support element 1206, when rearward housing portion 1204 and support element 1206 are sealed together, as by ultrasonic sealing, with forwardly facing edge 1242 of rearward housing portion 1204 abutting ring surface 1268 of support element 1206.

In accordance with an embodiment of the present invention, as seen in FIG. 12A, bacterial filter assembly 1300 is preferably formed of a plurality of mutually spaced, generally parallel elongate hollow fibers 1303, which are potted at both ends thereof so as to define respective forward and rearward potted ends 1304 and 1306.

The hollow fibers 1303 are preferably formed of polysulfone. A preferred hollow fiber is CFP-2-E, commercially available from GE Healthcare of Pittsburgh, Pa., USA. In accordance with an embodiment of the present invention, each hollow fiber is treated so as to have a hydrophobic strip 1308 extending generally along its entire length and defining a hydrophobic region. The hydrophobic strip 1308 is preferably realized by applying to the hollow fiber a hydrophobic material, such as a siliconizing agent, for example MDX4®, commercially available from Dow Corning of Midland, Mich., USA. Following suitable curing of the hollow fibers 1303, the hollow fibers 1303 are arranged in a desired mutually spaced parallel arrangement and potted as shown in FIG. 12A. Preferably the potting is such that the forward ends of the hollow fibers 1303 are open and the rearward ends of the hollow fibers 1303 are sealed to define filter assembly 1300.

As seen in FIG. 13, the filter assembly 1300 is preferably located within interior volume 1302 and located as desired by engagement with shoulders 1246 and 1248 and is preferably fixed thereto, as by ultrasonic or RF sealing, which separate an upstream portion 1310 of interior volume 1302 from a downstream portion 1312 of interior volume 1302. It is seen that aperture 1252 of support element 1206 communicates with fluid flow passageway 1292 of valve 1280 and with upstream portion 1310 of interior volume 1302 and thus with the interiors of hollow fibers 1303. The exterior of hollow fibers 1303 communicates with the downstream portion 1312 of interior volume 1302 and with a fluid passageway defined by an interior bore of male luer 1230.

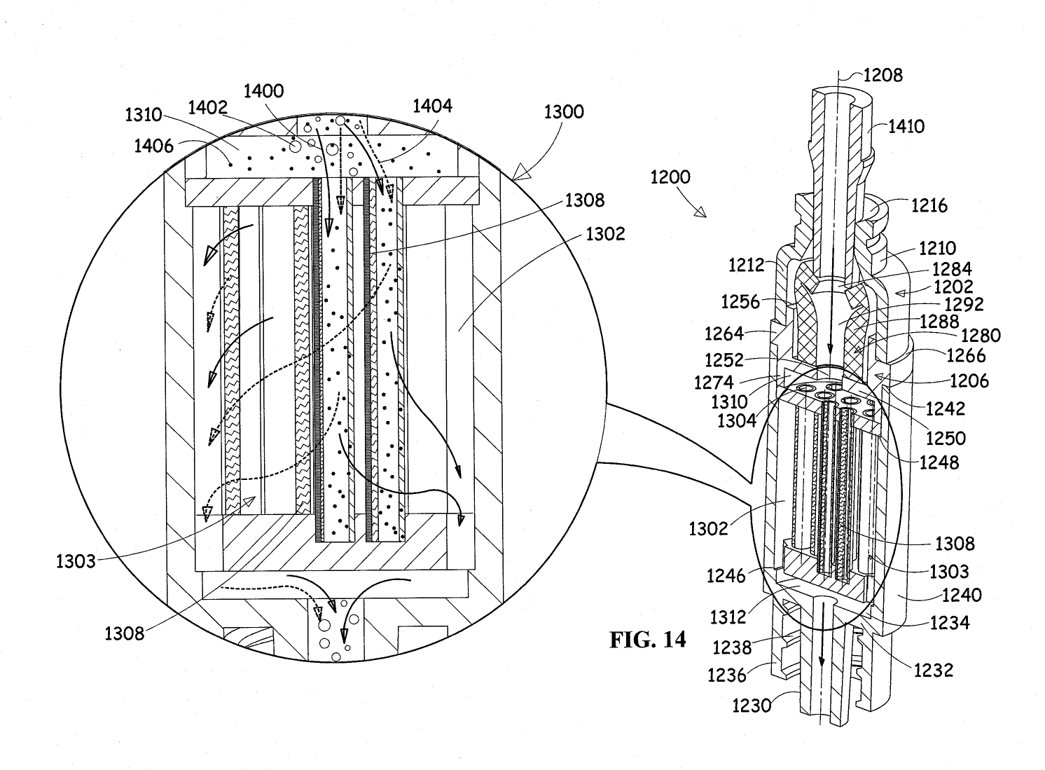

Reference is now made to FIG. 14, which is a detailed simplified, partially symbolic, side view sectional illustration of the operation of the bacterial filter assembly of FIG. 13. In FIG. 14, for ease of understanding, liquid flows are shown by solid arrows, here designated by reference numeral 1400, air bubbles are designated by reference numeral 1402, flows of air bubbles are shown by dashed arrows, here designated by reference numeral 1404, and bacteria are symbolized by small dots, here designated by reference numeral 1406.

When a male luer 1410 is inserted at the forward end of the bacteria-retaining medical valve 1200, engaging opening 1216 and bore 1218 of forward housing portion 1202, and thereby opening slit 1284, fluid, such as an infusion solution in which both air bubbles 1402 and bacteria 1406 may be present, flows through the male luer 1410 and passageway 1292 of valve 1280 via aperture 1252 and upstream portion 1310 of interior volume 1302 to the interiors of the hollow fibers 1303 of bacterial filter assembly 1300. The bacteria 1406 are retained in the hollow fibers 1303. Air bubbles 1402 pass through the hydrophobic strip 1308 of the hollow fibers 1303, which define hydrophobic bacterial impermeable passageways, and liquid passes through the remaining regions of the hollow fibers 1303, which are hydrophilic and which define hydrophilic bacterial impermeable passageways.

It is appreciated that in this embodiment of the present invention, at least an upstream part of bacterial filter assembly 1300, such as the forward portions of the hollow fibers 1303, reduces the pressure of liquid reaching at least a downstream part of the bacterial filter assembly 1300, such as the rearward portions of the hollow fibers 1303.

Thus it is understood that at least one pressure reducing passageway substantially reduces the pressure of liquid reaching at least some of the hydrophilic bacterial impermeable passageways downstream thereof.

It is a particular feature of the present invention that at least some of the hydrophobic bacterial impermeable passageways are located so as to prevent air buildup within the bacterial filter assembly 1300 at those hydrophilic bacterial impermeable passageways at which the liquid pressure is substantially reduced, which would otherwise block liquid flow therethrough.

It will be appreciated by persons skilled in the art that the present invention is not limited by what has been particularly shown and described hereinabove. Rather the scope of the present invention includes both combinations and subcombinations of various features described hereinabove as well as variations and modifications thereof which are not in the prior art.

1. A bacteria-retaining medical valve comprising:
a housing defining a liquid inlet and a liquid outlet;
a valve located within said housing downstream of said liquid inlet; and a bacterial filter assembly located within said housing downstream of said valve and upstream of said liquid outlet,
said bacterial filter assembly including:
hydrophilic bacterial impermeable passageways which are configured to permit liquid passage therethrough and prevent bacterial passage therethrough and air passage therethrough; and
hydrophobic bacterial impermeable passageways which are configured to permit air passage therethrough and prevent bacterial passage therethrough and liquid passage therethrough.

2. A bacteria-retaining medical valve according to claim 1 and wherein:
at least an upstream part of said bacterial filter assembly reduces the pressure of liquid reaching at least a downstream part of said bacterial filter assembly; and
at least some of said hydrophobic bacterial impermeable passageways are located so as to prevent air buildup within said bacterial filter assembly at at least some of said hydrophilic bacterial impermeable passageways at which the liquid pressure is substantially reduced, which would otherwise block liquid flow therethrough.

3. A bacteria-retaining medical valve according to claim 1 and wherein said hydrophobic bacterial impermeable passageways are formed in an air vent filter which is upstream of said hydrophilic bacterial impermeable passageways.

4. A bacteria-retaining medical valve according to claim 1 and wherein said bacterial filter assembly comprises a folded bacterial filter element which, when folded, has multiple, non co-planar liquid and air passage surfaces.

5. A bacteria-retaining medical valve according to claim 1 and wherein said bacterial filter assembly comprises a spiral wound filter element and spacer.

6. A bacteria-retaining medical valve according to claim 1 and wherein said bacterial filter assembly comprises an envelope formed of a filter substrate which encloses a spacer and is formed with a single aperture.

7. A bacteria-retaining medical valve according to claim 1 and wherein said bacterial filter assembly comprises a bundle of mutually spaced hollow fibers.

8. A bacteria-retaining medical filter comprising:
a housing defining a liquid inlet and a liquid outlet; and
a bacterial filter assembly located within said housing downstream of said liquid inlet and upstream of said liquid outlet,
said bacterial filter assembly including:
hydrophilic bacterial impermeable passageways which are configured to permit liquid passage therethrough and prevent bacterial passage therethrough and air passage therethrough; and
hydrophobic bacterial impermeable passageways which are configured to permit air passage therethrough and prevent bacterial passage therethrough and liquid passage therethrough, and wherein:
at least an upstream part of said bacterial filter assembly reduces the pressure of liquid reaching at least a downstream part of said bacterial filter assembly; and
at least some of said hydrophobic bacterial impermeable passageways are located so as to prevent air buildup within said bacterial filter assembly at at least some of said hydrophilic bacterial impermeable passageways at which the liquid pressure is substantially reduced, which would otherwise block liquid flow therethrough.

9. A bacteria-retaining medical filter according to claim 8 and wherein said bacterial filter assembly comprises a folded bacterial filter element which, when folded, has multiple, non co-planar liquid and air passage surfaces.

10. A bacteria-retaining medical filter according to claim 8 and wherein said bacterial filter assembly comprises a spiral wound filter element and spacer.

11. A bacteria-retaining medical filter according to claim 8 and wherein said bacterial filter assembly comprises an envelope formed of a filter substrate which encloses a spacer and is formed with a single aperture.

12. A bacteria-retaining medical filter according to claim 8 and wherein said bacterial filter assembly comprises a bundle of mutually spaced hollow fibers.

13. A bacteria-retaining medical valve comprising:
a housing defining a liquid inlet and a liquid outlet;
a valve located within said housing downstream of said liquid inlet; and a bacterial filter assembly located within said housing downstream of said valve and upstream of said liquid outlet,
said bacterial filter assembly being formed of a plurality of hollow fibers and including:
hydrophilic bacterial impermeable passageways which are configured to permit liquid passage therethrough and prevent bacterial passage therethrough and air passage therethrough; and
hydrophobic bacterial impermeable passageways which are configured to permit air passage therethrough and prevent bacterial passage therethrough and liquid passage therethrough.

14. A bacteria-retaining medical valve according to claim 13 and wherein: at least an upstream part of said bacterial filter assembly reduces the pressure of liquid reaching at least a downstream part of said bacterial filter assembly; and
at least some of said hydrophobic bacterial impermeable passageways are located so as to prevent air buildup within said bacterial filter assembly at at least some of said hydrophilic bacterial impermeable passageways at which the liquid pressure is substantially reduced, which would otherwise block liquid flow therethrough.

15-19. (canceled)
